(19) United States
(12) Patent Application Publication
(10) Pub. No.: US 2024/0423533 A1
(43) Pub. Date: Dec. 26, 2024

(54) SLEEP DETECTION USING CARDIAC AND RESPIRATION INFORMATION

(71) Applicant: Cardiac Pacemakers, Inc., St. Paul, MN (US)

(72) Inventors: Mojgan Goftari, Shoreview, MN (US); Kevin Chu, Shoreview, MN (US); John D. Hatlestad, Maplewood, MN (US); Pramodsingh Hirasingh Thakur, Woodbury, MN (US)

(21) Appl. No.: 18/752,499

(22) Filed: Jun. 24, 2024

Related U.S. Application Data
(60) Provisional application No. 63/523,158, filed on Jun. 26, 2023.

(51) Int. Cl. A61B 5/00; A61B 5/0245; A61B 5/08

(52) U.S. Cl. CPC A61B 5/4818; A61B 5/0245; A61B 5/0816; A61B 5/4809; A61B 5/4812; A61B 5/7264

(57) ABSTRACT

Systems and methods for monitoring and staging sleep using cardiac and respiration information are disclosed. An exemplary system comprises a storage device to store a trained hybrid sleep detection and classification model that comprise a plurality of trained machine-learning models each trained to map input cardiac and respiratory data into one of multiple distinct model-specific awake or sleep classes, and a trained regression model to combine the model-specific awake or sleep classes to a composite awake or sleep classification. A sleep detector applies patient cardiac and respiratory information to the trained hybrid sleep detection and classification model to determine a composite awake or sleep classification for the patient. The composite awake or sleep classification is used to diagnose medical conditions including sleep apnea.

### CLAIM OF PRIORITY

This application claims the benefit of U.S. Provisional Application No. 63/523,158 filed on Jun. 26, 2023, which is hereby incorporated by reference in its entirety.

### TECHNICAL FIELD

This document relates generally to medical devices, and more particularly, to systems, devices and methods for detecting and staging sleep using cardiac and respiration information.

### BACKGROUND

Detecting awake or sleep state and determining a sleep stage are useful in a variety of medical contexts. For example, sleep information have been used to diagnose certain medical conditions such as sleep apnea. Sleep apnea, one of common comorbidities of various heart diseases, generally involves a brief cessation of breathing during sleep. The most common type of sleep apnea is central sleep apnea. Central sleep apnea (CSA) is associated with the failure of the body to automatically initiate and control a respiratory cycle at the proper time. CSA typically causes cessation of substantially all respiratory effort during sleep. CSA may be developed after a heart attack, and is usually a contributing factor to heart failure and other cardiopulmonary disorders. Obstructive sleep apnea (OSA) is associated with a blockage of the airway, and is generally characterized by repetitive pauses in breathing during sleep due to upper airway obstruction or collapse. OSA is commonly found in overweight people who snore or have oversized necks. When awake, muscle tone keeps the throat open. When asleep, the airway of the neck narrows and closes. The person struggles to breathe against the collapsed throat as if choking. As the patient wakes up, the muscles of the throat open the airway. Many patients with congestive heart failure (CHF) suffer from obstructive sleep apnea. In addition to sleep apnea, sleep information may also be used to diagnose various psychological disorders such as depression and mania, among other context-aware diagnoses.

Implantable medical devices (IMDs) have been used for monitoring patient health condition or disease states and delivering therapies. For example, implantable cardioverter-defibrillators may be used to monitor for certain abnormal heart rhythms, such as bradycardia or tachycardia, and to deliver electrical energy to the heart to correct the abnormal rhythms. Some IMDs may be used to monitor for chronic worsening of cardiac hemodynamic performance, such as due to congestive heart failure (CHF), and to provide cardiac stimulation therapies, including cardiac resynchronization therapy (CRT) to correct cardiac dyssynchrony within a ventricle or between ventricles.

Certain cardiac diseases such as CHF can be worsened by excessive stress during apnea. Early and accurate detection of sleep apnea can be clinically important for assessing patient cardiac function and help prevent or slow worsening of certain heart conditions. Robust detection and staging of sleep can improve sleep apnea diagnosis and other context-aware diagnosis requiring a confirmed sleep state in a patient.

### Overview

Sleep apnea detection generally involves physiological measurement during a sleep study typically performed in a sleep lab. Physiological measurements can be collected during a confirmed sleep state, including, for example, respiration rate, tidal volume, respiration flow, or a respiration pattern, or indirect respiration measurement such as transthoracic impedance, acoustic signals indicative of respiration, or other respiration surrogates. However, a sleep study can be less convenient and/or less comfortable for some patients. Patients may also be constrained by accessibility, scheduling, and cost associated with a sleep study. Early detection of apnea events and stratifying an apnea risk is generally desired.

Sleep detection and characterization has primarily relied on the electroencephalogram (EEG), where an array of electrodes are placed on a patient's scalp and coupled to an external monitoring device. Collecting and analyzing EEG and implementing an EEG-based sleep detection are generally performed in a clinical setting, and it can be challenging to perform outside the clinic such as in a patient's home. Existing ambulatory medical device, such as implantable medical devices (IMDs) or implantable cardiac monitors (ICMs), include sensors for sensing cardiac and respiratory information, but generally lack sensors for sensing and capabilities of processing EEG signals. Existing EEG-based sleep detection techniques are generally unsuitable for long-term in-home monitoring of the patient's sleep state and sleep apnea diagnosis.

The present inventors have recognized an unmet need for apparatus and techniques to more conveniently and effectively detect sleep state in an ambulatory or in-home setting without requiring EEG or other brain signals. The present document describes systems, devices, and methods for detecting and staging sleep using cardiac and respiration information and a machine learning (ML)-based sleep detection model. An exemplary system comprises a storage device to store a trained hybrid sleep detection and classification model that comprise a plurality of trained ML models each trained to map input cardiac and respiratory data into one of multiple distinct model-specific awake or sleep classes, and a trained regression model to combine the model-specific awake or sleep classes from the plurality of trained ML models into a composite awake or sleep classification. A sleep detector applies patient cardiac and respiratory information to the trained hybrid sleep detection and classification model to determine a composite awake or sleep classification for the patient. The composite awake or sleep classification may be used in context-aware diagnosis of various diseases or conditions including, but not limited to, sleep apnea.

Example 1 is a medical-device system for monitoring and staging sleep in a patient, the system comprising: a receiver circuit configured to receive cardiac information and respiratory information of the patient; a storage device configured to store a trained hybrid sleep detection and classification model comprising: a plurality of trained machine-learning (ML) models each trained to map cardiac and respiratory data into one of multiple distinct model-specific awake or sleep classes; and a trained regression model trained to map the model-specific awake or sleep classes produced by the plurality of ML models to a composite awake or sleep classification; and a controller circuit, comprising a sleep detector configured to apply the received cardiac and respiratory information to the trained hybrid sleep detection and classification model to determine a corresponding composite awake or sleep classification for the patient; wherein the controller is configured to provide the determined composite awake or sleep classification to a user or a process executable by the medical-device system.

In Example 2, the subject matter of Example 1 optionally includes, wherein to apply the received cardiac and respiratory information to the trained hybrid sleep detection and classification model to determine the corresponding composite awake or sleep classification, the controller circuit is configured to: apply the received cardiac and respiratory information to each of the plurality of trained ML models to determine a model-specific awake or sleep classification and a confidence score associated with the determined model-specific awake or sleep classification; and apply the confidence scores associated with the determined awake or sleep classes to the trained regression model to determine the corresponding composite awake or sleep classification for the patient.

In Example 3, the subject matter of any one or more of Examples 1-2 optionally includes the receiver circuit that can be electrically coupled to a cardiac sensor configured to sense the cardiac information of the patient including a surface or subcutaneous electrocardiogram or heart sound information.

In Example 4, the subject matter of any one or more of Examples 1-3 optionally includes the receiver circuit that can be electrically coupled to a respiratory sensor configured to sense the respiratory information of the patient including a respiratory rate or a tidal volume.

In Example 5, the subject matter of Example 4 optionally includes the plurality of trained ML models that can include binary classification models each trained to map the cardiac and respiratory data into one of two model-specific awake or sleep classes.

In Example 6, the subject matter of any one or more of Examples 1-5 optionally includes the plurality of trained ML models that can include one or more trained decision tree models.

In Example 7, the subject matter of any one or more of Examples 1-6 optionally includes the plurality of trained ML models that can include one or more trained random forest models.

In Example 8, the subject matter of any one or more of Examples 1-7 optionally includes the plurality of trained ML models that can include one or more neural network or deep neural network models.

In Example 9, the subject matter of any one or more of Examples 1-8 optionally includes the model-specific awake or sleep classes that can include one or more of an awake state or a sleep state.

In Example 10, the subject matter of any one or more of Examples 1-9 optionally includes the model-specific awake or sleep classes that can include one or more sleep phases or stages selected from the group consisting of: a rapid eye movement (REM) phase of sleep; a non-REM phase of sleep; an N1 stage of non-REM sleep; an N2 stage of non-REM sleep; a combined N1-N2 stage of non-REM sleep; and an N3 stage of non-REM sleep.

In Example 11, the subject matter of any one or more of Examples 1-10 optionally includes the trained regression model that can be trained to determine respective weights for confidence scores associated with the determined awake or sleep classes, and to determine the composite awake or sleep classification using a weighted combination of the confidence scores each weighted by the respective weights.

In Example 12, the subject matter of any one or more of Examples 1-11 optionally includes the trained regression model that can be a logistic regression model.

In Example 13, the subject matter of any one or more of Examples 1-12 optionally includes he controller circuit that can include a training module configured to generate the trained hybrid sleep detection and classification model, including: to generate the plurality of trained ML models using a first training dataset comprising input cardiac and respiratory data collected from a group of patients during known awake or sleep states or sleep stages; and to generate the trained regression model using a second training dataset comprising awake or sleep classifications and confidence scores associated with the awake or sleep classifications and the known awake or sleep states or sleep stages.

In Example 14, the subject matter of any one or more of Examples 1-13 optionally includes an apnea detector circuit configured to detect sleep apnea during a time when the composite awake or sleep classification satisfies a specific sleep phase or stage requirement.

Example 15 is a method of monitoring and staging sleep in a patient using a medical-device system, the method comprising: receiving cardiac information and respiratory information of the patient; generating and storing in a storage device a trained hybrid sleep detection and classification model comprising (i) a plurality of trained machine-learning (ML) models each trained to map cardiac and respiratory data into one of multiple distinct model-specific awake or sleep classes, and (ii) a trained regression model trained to map the model-specific awake or sleep classes produced by the plurality of ML models to a composite awake or sleep classification; applying, via a sleep detector, the received cardiac and respiratory information to the trained hybrid sleep detection and classification model to determine a corresponding composite awake or sleep classification for the patient; and providing the determined composite awake or sleep classification to a user or a process executable by the medical-device system.

In Example 16, the subject matter of Example 15 optionally includes applying the received cardiac and respiratory information to the trained hybrid sleep detection and classification model to determine the corresponding composite awake or sleep classification, which can include: applying the received cardiac and respiratory information to each of the plurality of trained ML models to determine a model-specific awake or sleep classification and a confidence score associated with the determined model-specific awake or sleep classification; and applying the confidence scores associated with the determined awake or sleep classes to the trained regression model to determine the corresponding composite awake or sleep classification for the patient.

In Example 17, the subject matter of any one or more of Examples 15-16 optionally includes the trained regression model that can be trained to determine respective weights for confidence scores associated with the determined awake or sleep classes, and to determine the composite awake or sleep classification using a weighted combination of the confidence scores each weighted by the respective weights.

In Example 18, the subject matter of any one or more of Examples 15-17 optionally includes the received cardiac information that can include a surface or subcutaneous electrocardiogram or heart sound information; and the received respiratory information that can include a respiratory rate or a tidal volume.

In Example 19, the subject matter of any one or more of Examples 15-18 optionally includes the plurality of trained ML models that can include binary classification models each trained to map the cardiac and respiratory data into one of two model-specific awake or sleep classes.

In Example 20, the subject matter of any one or more of Examples 15-19 optionally includes the plurality of trained ML models that can include one or more trained decision tree models, one or more trained random forest models, or one or more neural network or deep neural network models.

In Example 21, the subject matter of any one or more of Examples 15-20 optionally includes the model-specific awake or sleep classes that can include one or more of: an awake state; a sleep state; a rapid eye movement (REM) phase of sleep; a non-REM phase of sleep; an N1 stage of non-REM sleep; an N2 stage of non-REM sleep; a combined N1-N2 stage of non-REM sleep; and an N3 stage of non-REM sleep.

In Example 22, the subject matter of any one or more of Examples 15-21 optionally includes an apnea detector circuit configured to detect sleep apnea during a time when the composite awake or sleep classification satisfies a specific sleep phase or stage requirement.

The systems, devices, and methods discussed in this document may improve the medical technology of automated device-based sleep detection which can be further be used to improve diagnosis of certain diseases or conditions such as sleep apnea. Compared to conventional EEG-based sleep detection, the cardiac and respiratory information-based sleep detection and staging as described herein can be implemented in an implantable or wearable device to provide convenient yet effective sleep detection using relatively simple and low cost sensors already implemented in the implantable or wearable device. The hybrid sleep detection and classification model as described herein advantageously integrate a group of trained ML-based binary sleep classification models with a subsequent regression model as a fusion engine to combine the multiple binary classification results into a composite sleep state decision. Such hybrid detection and classification architecture may improve sleep detection specificity and enhance detector robustness against noise and interferences which are typically encountered by conventional sleep detectors. The improved sleep detection allows for more accurate diagnosis and characterization of sleep apnea or other conditions requiring a confirmed sleep state in a patient. Additionally, compared to EEG-based sleep detection, the present cardiac and respiratory information-based sleep detection has a lower complexity and reduced implementation cost, and uses less obtrusive systems, apparatus, and methods. It may particularly be beneficial for patients at early stage of sleep apnea or those constrained by accessibility, scheduling, affordability, or various contraindications to sleep studies.

The cardiac and respiratory information-based sleep detection and classification using a hybrid detection model as described in this disclosure can also improve power and resource usage in a medical device of system. A technological problem exists in medical devices and medical device systems that in low-power monitoring modes, ambulatory medical devices powered by one or more rechargeable or non-rechargeable batteries (e.g., including IMDs) have to make certain tradeoffs between battery life, or in the instance of implantable medical devices with non-rechargeable batteries, between device replacement periods often including surgical procedures, and sampling resolution, sampling periods, of processing, storage, and transmission of sensed physiologic information, or features or mode selection of or within the medical devices. Medical devices can include higher-power modes and lower-power modes. Physiologic information, such as indicative of a potential adverse physiologic event, can be used to transition from a low-power mode to a high-power mode. In certain examples, the low-power mode can include a low resource mode, characterized as requiring less power, processing time, memory, or communication time or bandwidth (e.g., transferring less data, etc.) than a corresponding high-power mode. The high-power mode can include a relatively higher resource mode, characterized as requiring more power, processing time, memory, or communication time or bandwidth than the corresponding low-power mode. However, by the time physiologic information detected in the low-power mode indicates a possible event, valuable information has been lost, unable to be recorded in the high-power mode. The inverse is also true, in that false or inaccurate determinations that trigger a high-power mode unnecessarily unduly limit the usable life of certain ambulatory medical devices. The cardiac and respiratory information-based sleep detection and classification as described herein allows for more accurate and efficient detection and staging of sleep, such that certain patient monitoring functionalities or devices (e.g., sensors) can be activated or deactivated depending on patient awake or sleep state or certain sleep phases or stages. Accordingly, certain unnecessary transitions from the low-power mode to the high-power mode can be avoided, and use of medical device resources and power can be improved.

This Overview is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates generally an example of a patient management system and portions of an environment in which the system may operate.

FIG. 2 illustrates generally an example of a sleep detection and staging system for monitoring and staging sleep using patient cardiac and respiratory information.

FIG. 3 illustrates generally an example of a trained hybrid sleep detection and classification model to detect a detect a sleep or awake state or sleep stage.

FIGS. 4A-4B illustrate example confusion matrices showing sleep classification performances of a single sleep detection model and a hybrid sleep detection model.

FIGS. 5A-5B are graphs of receiver operating characteristic (ROC) curves showing sleep apnea detection performances with and without confirmed sleep state of the patient.

FIG. 6 is a flowchart illustrating an example method of monitoring and staging sleep in a patient.

FIG. 7 illustrates generally a block diagram of an example machine upon which any one or more of the techniques (e.g., methodologies) discussed herein may perform.

### DETAILED DESCRIPTION

Disclosed herein are systems, devices, and methods for detecting and staging sleep using cardiac and respiration information. An exemplary system comprises a storage device to store a trained hybrid sleep detection and classification model comprising a plurality of trained machine-learning (ML) models and a trained regression model. The ML models are each trained to map input cardiac and respiratory data into one of multiple distinct model-specific awake or sleep classes. The regression model is trained to combine the model-specific awake or sleep classes into a composite awake or sleep classification. The system includes a sleep detector that applies cardiac and respiratory information sensed from a patient to the trained hybrid sleep detection and classification model to determine a composite awake or sleep classification for the patient. The composite awake or sleep classification can be provided to a user or a process executable by the medical-device system, such as for diagnosing sleep apnea or other medical conditions or adjusting a therapy.

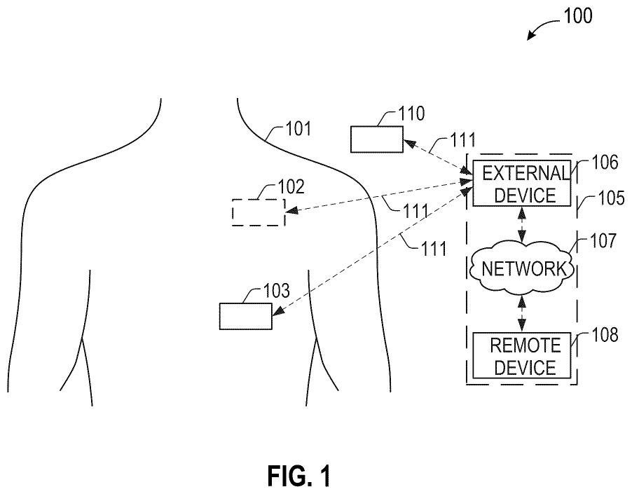

FIG. 1 illustrates an example patient management system 100 and portions of an environment in which the patient management system 100 may operate. The patient management system 100 can perform a range of activities, including remote patient monitoring and diagnosis of a disease condition. Such activities can be performed proximal to a patient 101, such as in a patient home or office, through a centralized server, such as in a hospital, clinic, or physician office, or through a remote workstation, such as a secure wireless mobile computing device.

The patient management system 100 can include one or more ambulatory medical devices, an external system 105, and a communication link 111 providing for communication between the one or more ambulatory medical devices and the external system 105. The one or more ambulatory medical devices can include an implantable medical device (IMD) 102, a wearable medical device (WMD) 103, or one or more other implantable, leadless, subcutaneous, external, wearable, or ambulatory medical devices configured to monitor, sense, or detect information from, determine physiological information about, or provide one or more therapies to treat various conditions of the patient 101, such as one or more cardiac or non-cardiac conditions (e.g., dehydration, sleep disordered breathing, etc.).

In an example, the IMD 102 can include one or more traditional cardiac rhythm management devices implanted in a chest of a patient, having a lead system including one or more transvenous, subcutaneous, or non-invasive leads or catheters to position one or more electrodes or other sensors (e.g., a heart sound sensor) in, on, or about a heart or one or more other position in a thorax, abdomen, or neck of the patient 101. In another example, the IMD 102 can include a monitor implanted, for example, subcutaneously in the chest of patient 101, the IMD 102 including a housing containing circuitry and, in certain examples, one or more sensors, such as a temperature sensor, etc.

The IMD 102 can include an assessment circuit configured to detect or determine specific physiological information of the patient 101, or to determine one or more conditions or provide information or an alert to a user, such as the patient 101 (e.g., a patient), a clinician, or one or more other caregivers or processes. In an example, the IMD 102 can be an implantable cardiac monitor (ICM) configured to collected cardiac information, optionally along with other physiological information, from the patient. The IMD 102 can alternatively or additionally be configured as a therapeutic device configured to treat one or more medical conditions of the patient 101. The therapy can be delivered to the patient 101 via the lead system and associated electrodes or using one or more other delivery mechanisms. The therapy can include delivery of one or more drugs to the patient 101, such as using the IMD 102 or one or more of the other ambulatory medical devices, etc. In some examples, therapy can include cardiac resynchronization therapy for rectifying dyssynchrony and improving cardiac function in heart failure patients. In other examples, the IMD 102 can include a drug delivery system, such as a drug infusion pump to deliver drugs to the patient for managing arrhythmias or complications from arrhythmias, hypertension, or one or more other physiologic conditions. In other examples, the IMD 102 can include one or more electrodes configured to stimulate the nervous system of the patient or to provide stimulation to the muscles of the patient airway, etc.

The WMD 103 can include one or more wearable or external medical sensors or devices (e.g., automatic external defibrillators (AEDs), Holter monitors, patch-based devices, smart watches, smart accessories, wrist- or finger-worn medical devices, such as a finger-based photoplethysmography sensor, etc.).

The external system 105 can include a dedicated hardware/software system, such as a programmer, a remote server-based patient management system, or alternatively a system defined predominantly by software running on a standard personal computer. The external system 105 can manage the patient 101 through the IMD 102 or one or more other ambulatory medical devices connected to the external system 105 via a communication link 111. In other examples, the IMD 102 can be connected to the WMD 103, or the WMD 103 can be connected to the external system 105, via the communication link 111. This can include, for example, programming the IMD 102 to perform one or more of acquiring physiological data, performing at least one self-diagnostic test (such as for a device operational status), analyzing the physiological data, or optionally delivering or adjusting a therapy for the patient 101. Additionally, the external system 105 can send information to, or receive information from, the IMD 102 or the WMD 103 via the communication link 111. Examples of the information can include real-time or stored physiological data from the patient 101, diagnostic data, such as detection of patient hydration status, hospitalizations, responses to therapies delivered to the patient 101, or device operational status of the IMD 102 or the WMD 103 (e.g., battery status, lead impedance, etc.). The communication link 111 can be an inductive telemetry link, a capacitive telemetry link, or a radiofrequency (RF) telemetry link, or wireless telemetry based on, for example, “strong” Bluetooth or IEEE 802.11 wireless fidelity “Wi-Fi” interfacing standards. Other configurations and combinations of patient data source interfacing are possible.

The external system 105 can include an external device 106 in proximity of the one or more ambulatory medical devices, and a remote device 108 in a location relatively distant from the one or more ambulatory medical devices, in communication with the external device 106 via a communication network 107. Examples of the external device 106 can include a medical device programmer. The remote device 108 can be configured to evaluate collected patient or patient information and provide alert notifications, among other possible functions. In an example, the remote device 108 can include a centralized server acting as a central hub for collected data storage and analysis. The server can be configured as a uni-, multi-, or distributed computing and processing system. The remote device 108 can receive data from multiple patients. The data can be collected by the one or more ambulatory medical devices, among other data acquisition sensors or devices associated with the patient 101. The server can include a memory device to store the data in a patient database. The server can include an alert analyzer circuit to evaluate the collected data to determine if specific alert condition is satisfied. Satisfaction of the alert condition may trigger a generation of alert notifications, such to be provided by one or more human-perceptible user interfaces. In some examples, the alert conditions may alternatively or additionally be evaluated by the one or more ambulatory medical devices, such as the implantable medical device. By way of example, alert notifications can include a Web page update, phone or pager call, E-mail, SMS, text or “Instant” message, as well as a message to the patient and a simultaneous direct notification to emergency services and to the clinician. Other alert notifications are possible. The server can include an alert prioritizer circuit configured to prioritize the alert notifications. For example, an alert of a detected physiological event can be prioritized using a similarity metric between the physiological data associated with the detected physiological event to physiological data associated with the historical alerts.

The remote device 108 may additionally include one or more locally configured clients or remote clients securely connected over the communication network 107 to the server. Examples of the clients can include personal desktops, notebook computers, mobile devices, or other computing devices. System users, such as clinicians or other qualified medical specialists, may use the clients to securely access stored patient data assembled in the database in the server, and to select and prioritize patients and alerts for health care provisioning. In addition to generating alert notifications, the remote device 108, including the server and the interconnected clients, may also execute a follow-up scheme by sending follow-up requests to the one or more ambulatory medical devices, or by sending a message or other communication to the patient 101 (e.g., the patient), clinician or authorized third party as a compliance notification.

The communication network 107 can provide wired or wireless interconnectivity. In an example, the communication network 107 can be based on the Transmission Control Protocol/Internet Protocol (TCP/IP) network communication specification, although other types or combinations of networking implementations are possible. Similarly, other network topologies and arrangements are possible.

One or more of the external device 106 or the remote device 108 can output the detected physiological events to a system user, such as the patient or a clinician, or to a process including, for example, an instance of a computer program executable in a microprocessor. In an example, the process can include an automated generation of recommendations for anti-arrhythmic therapy, or a recommendation for further diagnostic test or treatment. In an example, the external device 106 or the remote device 108 can include a respective display unit for displaying the physiologic or functional signals, or alerts, alarms, emergency calls, or other forms of warnings to signal the detection of arrhythmias. In some examples, the external system 105 can include an external data processor configured to analyze the physiologic or functional signals received by the one or more ambulatory medical devices, and to confirm or reject the detection of arrhythmias. Computationally intensive algorithms, such as machine-learning algorithms, can be implemented in the external data processor to process the data retrospectively to detect cardia arrhythmias.

Portions of the one or more ambulatory medical devices or the external system 105 can be implemented using hardware, software, firmware, or combinations thereof. Portions of the one or more ambulatory medical devices or the external system 105 can be implemented using an application-specific circuit that can be constructed or configured to perform one or more functions or can be implemented using a general-purpose circuit that can be programmed or otherwise configured to perform one or more functions. Such a general-purpose circuit can include a microprocessor or a portion thereof, a microcontroller or a portion thereof, or a programmable logic circuit, a memory circuit, a network interface, and various components for interconnecting these components. For example, a “comparator” can include, among other things, an electronic circuit comparator that can be constructed to perform the specific function of a comparison between two signals or the comparator can be implemented as a portion of a general-purpose circuit that can be driven by a code instructing a portion of the general-purpose circuit to perform a comparison between the two signals. “Sensors” can include electronic circuits configured to receive information and provide an electronic output representative of such received information.

The therapy device 110 can be configured to send information to or receive information from one or more of the ambulatory medical devices or the external system 105 using the communication link 111. In an example, the one or more ambulatory medical devices, the external device 106, or the remote device 108 can be configured to control one or more parameters of the therapy device 110. The external system 105 can allow for programming the one or more ambulatory medical devices and can receives information about one or more signals acquired by the one or more ambulatory medical devices, such as can be received via a communication link 111. The external system 105 can include a local external implantable medical device programmer. The external system 105 can include a remote patient management system that can monitor patient status or adjust one or more therapies such as from a remote location.

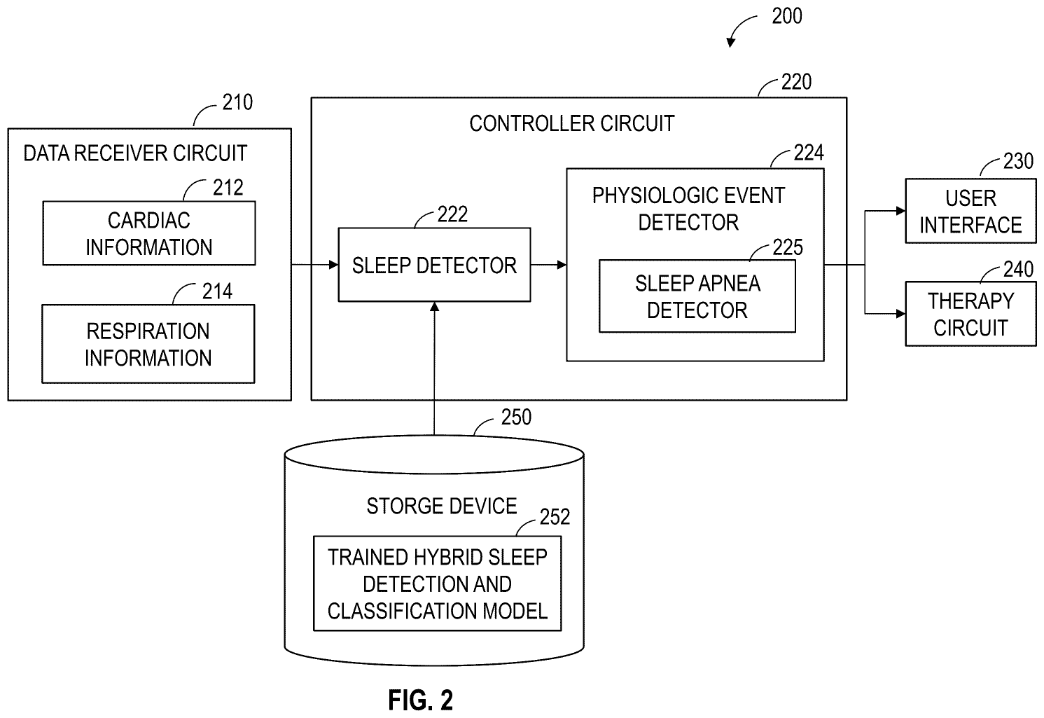

FIG. 2 illustrates generally an example of a sleep detection and staging system 200 for monitoring and staging sleep using at least cardiac and respiratory information sensed from a patient. Results of the sleep detection and staging can be used to detect physiological events such as sleep apnea or other context-aware diagnosis, or to adjust drug of device therapy. Portions of the sleep detection and staging system 200 may be implemented in one or more of the IMD 102, the WMD 103, or at least a portion of the external system 105.

The sleep detection and staging system 200 may include one or more of a data receiver circuit 210, a controller circuit 220, a user interface 230, a therapy circuit 240, and a storage device 250. The data receiver circuit 210 may receive physiological information from the patient. In an example, the data receiver circuit 210 may include circuitry to sense a physiologic signal from the patient via one or more implantable, wearable, or otherwise ambulatory sensors or electrodes associated with the patient. By way of example and not limitation and as illustrated in FIG. 2, the data receiver circuit 210 may receive cardiac information 212 and respiration information 214. Examples of the cardiac information include surface electrocardiography (ECG) subcutaneous ECG, or intracardiac electrogram (EGM), or cardiac mechanical information such as heart sounds (or cardiac acceleration), cardiac pressure including pulmonary artery pressure, left atrial pressure, RV pressure, LV coronary pressure, among others. The respiration information 214 may be sensed using a respiratory sensor which may be coupled to an implantable, wearable, holdable, or otherwise ambulatory respiratory sensor including one of an accelerometer, a microphone, an impedance sensor, or a flow sensor. Examples of the respiration information 214 include a tidal volume, a respiration rate, a minute ventilation, a respiratory sound, or a rapid-shallow breathing index (RSBI) computed as a ratio of a respiratory rate measurement to a tidal volume measurement, among others. In some examples, the respiration information 214 may include respiratory phase within a respiratory cycle. Examples of the respiratory phase may include an inspiration phase, an expiration phase, an apneic phase, a hypopneic phase, a transitional phase from inspiration to expiration, or a transitional phase from expiration to inspiration. The respiratory phase may include a specified portion of the inspiration, expiration, or transitional phase of respiration with respect to a reference time of respiration, such as end of inspiration or end of expiration.

The data receiver circuit 210 may receive other physiological or functional signals including, for example, physical activity signal, posture signal, a thoracic or cardiac impedance signal, arterial pressure signal, coronary blood temperature signal, blood oxygen saturation signal, heart sound signal, physiologic response to activity, apnea hypopnea index, one or more respiration signals such as a respiratory rate signal or a tidal volume signal, brain natriuretic peptide (BNP), blood panel, sodium and potassium levels, glucose level and other biomarkers and bio-chemical markers, among others. The data receiver circuit 210 may include one or more other sub-circuits to digitize, filter, or perform other signal conditioning operations on the received physiologic signal. In some examples, The data receiver circuit 210 may receive physiological information from a storage device such as an electronic medical record system that stores physiological information collected from the patient.

The controller circuit 220, communicatively coupled to the data receiver circuit 210, may detect and stage sleep using at least cardiac and respiratory information received from the data receiver circuit 210. The controller circuit 220 may be implemented as a part of a microprocessor circuit, which may be a dedicated processor such as a digital signal processor, application specific integrated circuit (ASIC), microprocessor, or other type of processor for processing information including physical activity information. Alternatively, the microprocessor circuit may be a general-purpose processor that may receive and execute a set of instructions of performing the functions, methods, or techniques described herein.

The controller circuit 220 may include circuit sets comprising one or more other circuits or sub-circuits, including a sleep detector 222 and a physiologic event detector 224. These circuits may, alone or in combination, perform the functions, methods, or techniques described herein. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

The sleep detector 222 can detect a sleep or awake state and determine a sleep stage using at least the cardiac information 212 and the respiration information 214. As shown in FIG. 2, the sleep detector 222 may be operatively in communication with the storage device 250 that stores a trained hybrid sleep detection and classification model 252. The sleep detector 222 may apply the cardiac and respiration information, or features derived therefrom, to the trained hybrid sleep detection and classification model 252 to determine a corresponding composite awake or sleep classification for the patient. Examples of the cardiac and respiration information may include respiration rate and tidal volume estimated from thoracic impedance measurements which are correlated to tidal volume, morphological, temporal, or statical features derived from raw ECG and respiration signals such as mean of upper and lower quartiles, minimum, maximum, variance, root-mean squared value, etc.

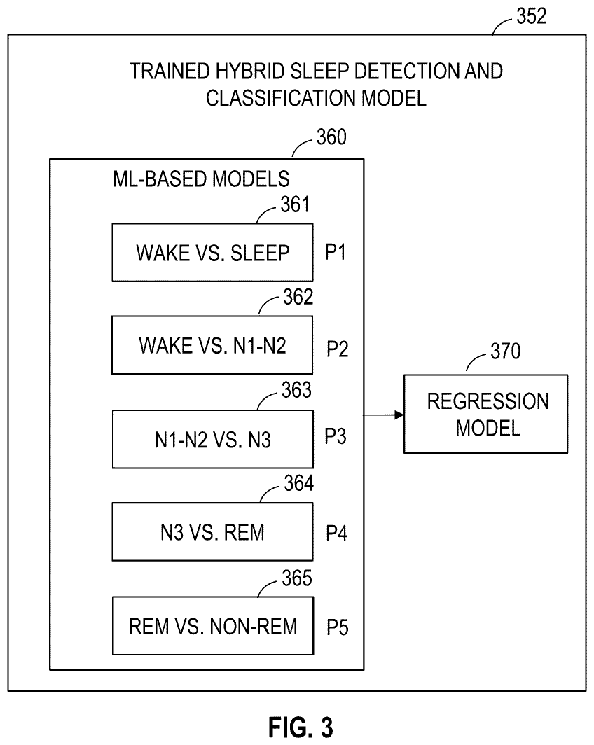

The trained hybrid sleep detection and classification model 252 may include a plurality of trained machine-learning (ML) models and a trained regression model. The trained ML models are each trained to map input cardiac and respiratory data into one of multiple distinct model-specific awake or sleep classes. The regression model is trained to combine the model-specific awake or sleep classes produced by the plurality of ML models into a composite awake or sleep classification. In various examples, the sleep detector 222 may determine that the patient is in one of the awake or sleep phases and sleep stages in a sleep cycle, including an awake state, a sleep state, a non-rapid eye movement (REM) phase of sleep, one or more of progressively deeper stages of non-REM sleep including an N1 stage of non-REM sleep, an N2 stage of non-REM sleep, a combined N1-N2 stage of non-REM sleep, and a N3 stage of non-REM sleep, and a REM phase of sleep. As non-REM stages progress, stronger stimuli are required to result in an awakening. N1 stage of non-REM sleep is the transition between wakefulness and sleep. It occurs upon falling asleep and during brief arousal periods within sleep. N2 stage of non-REM sleep occurs throughout the sleep period and represents 45-55% of total sleep time. N3 stage of non-REM sleep is also known as deep sleep or slow-wave sleep characterized by a particular pattern that appears in measurements of brain activity, and occurs mostly in the first third of the night and constitutes 10-20% of total sleep time. Examples of the hybrid sleep detection and classification model 252 are discussed below such as with respect to FIG. 3.

The ML models included in the trained hybrid sleep detection and classification model 252 may each have a specific architecture. In one example, at least some of the ML models may each have a decision tree structure. In another example, at least some of the ML models may each have a random forest (RF) structure. In yet another example, at least some of the ML models may each have a neural network structure comprising an input layer, one or more hidden layers, and an output layer. Patient data received by the data receiver circuit 210, including the cardiac information 212 and respiration information 214, can be provided to the input layer of each of the ML models. The ML models can propagate the input data through one or more hidden layers to the output layer. The ML models can provide the sleep detection and staging system 200 with the ability to perform tasks, without explicitly being programmed, by making inferences on awake or sleep state and sleep phases or stage based on patterns found in the analysis of cardiac information and respiratory information. The ML models explore the study and construction of algorithms (e.g., ML algorithms) that may learn from existing data and make predictions about new data. Such algorithms operate by building the ML models from training data in order to make data-driven predictions or decisions expressed as outputs or assessments.

The ML models may be trained using supervised learning or unsupervised learning. Supervised learning uses prior knowledge (e.g., examples that correlate inputs to outputs or outcomes) to learn the relationships between the inputs and the outputs. The goal of supervised learning is to learn a function that, given some training data, best approximates the relationship between the training inputs and outputs so that the ML model can implement the same relationships when given inputs to generate the corresponding outputs. Unsupervised learning is the training of an ML algorithm using information that is neither classified nor labeled, and allowing the algorithm to act on that information without guidance. Unsupervised learning is useful in exploratory analysis because it can automatically identify structure in data.

Some common tasks for unsupervised learning include clustering, representation learning, and density estimation. Some examples of commonly used unsupervised learning algorithms are K-means clustering, principal component analysis, and autoencoders. Some common tasks for supervised learning are classification problems and regression problems. Classification problems, also referred to as categorization problems, aim at classifying items into one of several category values. Regression algorithms aim at quantifying some items (for example, by providing a score to the value of some input). Some examples of commonly used supervised-ML algorithms are Logistic Regression (LR), Naive-Bayes, Decision tree, Random Forest (RF), neural networks (NN), deep neural networks (DNN), matrix factorization, and Support Vector Machines (SVM).

In an example, the ML models may be trained using deep learning. The ML model has an architecture of a deep neural network. Examples of DNN include a convolutional neural network (CNN), a recurrent neural network (RNN), a deep belief network (DBN), a long-term and short-term memory (LSTM) network, a transfer learning network, or a hybrid neural network comprising two or more neural network models of different types of different model configurations.

Another type of ML is collaborative learning that trains an algorithm across multiple decentralized devices holding local data, without exchanging the data. This approach stands in contrast to traditional centralized machine learning techniques where all the local datasets are uploaded to one server, as well as to more classical decentralized approaches which often assume that local data samples are identically distributed. Collaborative learning enables multiple actors to build a common, robust machine learning model without sharing data, thus allowing to address critical issues such as data privacy, data security, data access rights and access to heterogeneous data.

In some examples, the sleep detection and staging system 200 may include or otherwise communicate with a training module configured to train the ML models. To train an ML model, a training dataset can be constructed using patient information collected from a patient population each having respective AMDs of the same or similar type (e.g., implantable devices or wearable devices). The training data may include cardiac information and respiratory information, optionally along with other physiological information, collected by their respective AMDs, and assessments of awake or sleep states and sleep phases or stages (e.g., an awake state, a sleep state, a rapid eye movement (REM) phase of sleep, a non-REM phase of sleep, an N1 stage of non-REM sleep, an N2 stage of non-REM sleep, a combined N1-N2 stage of non-REM sleep, or a N3 stage of non-REM sleep). The assessments can be provided by a user (e.g., a clinician), or be made based on diagnostic tests using application-specific sensors or instruments. Such assessment of awake or sleep state and sleep phases or stages are output labels for training the ML models. The training of ML models may be performed continuously or periodically, or in near real time as additional patient information are received. The training involves algorithmically adjusting one or more ML model parameters, until the ML model being trained satisfies a specified training convergence criterion, such as the model outputs being as close as possible (within a specific margin) to the output labels.

The physiological event detector 224 can detect a physiological event, or make context-aware diagnosis using sleep information, including the sleep phases and/or stages (e.g., REM phase, non-REM phase, N1 stage of non-REM, N2-stage of non-REM, N1-N2 stage of non-REM, or N3 stage of non-REM) as identified by the sleep detector 222. For example, the sleep information may be used to detect sleep apnea, or diagnose psychological disorders such as depression or mania, among other medical conditions. In the illustrated example, the physiological event detector 224 may include an sleep apnea detector 225 to detect sleep apnea during a time when the patient is determined to be in a sleep state or in one or more specific sleep phases and/or stages (e.g., REM phase, non-REM phase, N1 stage of non-REM, N2-stage of non-REM, N1-N2 stage of non-REM, or N3 stage of non-REM), as detected by the sleep detector 222. Various techniques may be used to detect sleep apnea. In an example, the sleep apnea detector 225 can detect sleep apnea using respiration parameters, transthoracic impedance, acoustic signals indicative of respiration, or other respiration surrogates. In another example, the sleep apnea detector 225 can detect restrictive or obstructive respiratory conditions using heart sounds information, and further classify the detected restrictive or obstructive respiratory conditions as obstructive sleep apnea (OSA) or central sleep apnea (CSA) using hemodynamic parameters, optically along with other non-hemodynamic parameters, as described in the commonly assigned U.S. Pat. No. 10,799,187, entitled “SYSTEMS AND METHODS TO DETECT RESPIRATORY DISEASES,” which is hereby incorporated by reference in its entirety. The heart sounds, and hemodynamic and optional non-hemodynamic parameters, may be acquired during a time when the patient is determined to be in a sleep state or in one or more specific sleep stages. In yet another example, the sleep apnea detector 225 can determine a sleep apnea risk based on a bradycardia burden obtained during a time when the patient is determined to be in a sleep state or in one or more specific sleep stages. The bradycardia burden represents an accumulated time or relative time of the subject being in bradycardia over a specific monitoring period during sleep or a particular sleep stage. The apnea risk can be determined based on a comparison of the bradycardia burden to a threshold burden. The apnea risk can be represented by a numerical risk score directly proportional to the determined bradycardia burden, such that a higher bradycardia burden corresponds to a higher sleep apnea risk.

The user interface 330 may include an input device and an output device. In an example, at least a portion of the user interface 230 may be implemented in the external system 105. The input device may receive a user's programming input, such as parameters for cardiac and respiration information collection and processing and sleep detection. The input device may include a keyboard, on-screen keyboard, mouse, trackball, touchpad, touchscreen, or other pointing or navigating devices. The input device may enable a system user to program the parameters used for sensing the physiologic signals, detecting the arrhythmias, and generating alerts, among others. The output device may generate a human-perceptible presentation of. The output device may include a display for displaying cardiac and respiration information used for sleep detection and sleep apnea risk assessment, intermediate measurements or computations, and the apnea risk. The output unit may include a printer for printing hard copies of the detection information. The information may be presented in a table, a chart, a diagram, or any other types of textual, tabular, or graphical presentation formats. The presentation of the output information may include audio or other media format. The output unit may generate an alert to the system user or a recommendation of apnea evaluation or treatment in response to the determined apnea risk exceeding a risk threshold.

The therapy circuit 340 may be configured to deliver a therapy to the patient based on the detected physiological event such as sleep apnea. Examples of the therapy may include electrostimulation therapy delivered to the heart, a nerve tissue, other target tissues, a cardioversion therapy, a defibrillation therapy, or drug therapy. In some examples, the therapy circuit 340 may modify an existing therapy, such as adjust a stimulation parameter (e.g., a stimulation amplitude, frequency, pulse width, electrode configuration, stimulation time) of an electrostimulation therapy, or adjust a drug dosage of a drug therapy. The therapy can help prevent complications of sleep apnea or reduce apnea risk. In various examples, the therapy circuit 340 may adjust therapy based on the sleep phases and/or stages information produced by the sleep detector 222, such as timing and/or dosage of therapy. In one example, when the patient is detected to be asleep or in a particular sleep stage, the therapy circuit 340 may suspend drug therapy or device therapy (e.g., electrostimulation), or reduce intensity or dosage of the therapies. In another example, for a therapy intended to be delivered when the patient is asleep such as sleep apnea therapy to prevent or correct sleep apnea, the therapy circuit 340 may initiate or increase intensity or dosage of therapy based on a determination that the patient is asleep or at a specific sleep stage.

FIG. 3 illustrates generally an example of a trained hybrid sleep detection and classification model 352 to detect a sleep or awake state or sleep stage using at least cardiac information 212 and the respiration information 214. As an embodiment of the model 252 shown in FIG. 2, the trained hybrid sleep detection and classification model 352 may include a plurality of ML-based classification models 360 and a regression model 370. The ML-based classification models 354 each can be trained to map cardiac and respiratory data into a model-specific awake or sleep class. By way of example and not limitation, five ML-based classification models are shown: model 361 to discriminate an awake state from a sleep state; model 362 to discriminate an awake state from an N1-N2 stage of non-REM sleep; model 363 to discriminate an N1-N2 stage from an N3 stage of non-REM sleep; model 364 to discriminate an N3 stage of non-REM from a REM phase of sleep; and model 365 to discriminate a REM phase from a non-REM phase of sleep. Other numbers of ML-based classification models may be used. Although each of the ML-based classification models 361 through 365 are shown as binary classification model, in some examples, at least some of the ML-based classification models are multi-class (i.e., three or more classes) classification models.

As described above with respect to the trained hybrid sleep detection and classification model 252 in FIG. 2, the ML-based classification models 360 may each have a specific model architecture (e.g., a decision tree, a random forest, a neural network, a deep neural network, etc.). Each model can be trained using a training dataset that includes cardiac and respiratory information collected from a patient population during known awake or sleep states or sleep phases or stages. In some examples, cardiac and respiratory information during one sleep phase or stage may be used to train two or more different ML-based classification models. For example, since N2 stage is by definition also recognized as a “sleep state”, a “non-REM phase”, and a combined “N1-N2 stage” of non-REM sleep, cardiac and respiratory information collected from patients during a known N2 stage can also be labeled as a “sleep” class and used to train model 361, or labeled as “N1-N2 stage” to train each of models 362 and 363, or labeled as “non-REM” to train model 365.

In some examples, the ML-based classification models 360 may each be trained to output a confidence score (p) associated with the model-specific awake or sleep classification determined for the cardiac and respiration information input. The confidence score can be a probability (taking value between 0 and 1) indicating how likely the patient is in the classified state or sleep phase or stage. In an example, the ML-based classification models 360 are binary classification models each trained to produce, for a cardiac and respiration input, a corresponding confidence score p (0<p<1), where p=0 indicates a high confidence of one class, and p=1 indicates a high confidence of another class. For example, for model 361, p1=0 indicates a high confidence of patient being “awake”, p1=1 indicates a high confidence of a “sleep” state, and any value between 0 and 1 indicates a likelihood of being in a “sleep” state but not “awake” state. For example, p1=0.85 indicates a 85% probability that the patient is asleep, p1=0.1 indicates a 10% probability that the patient is asleep, or equivalently 90% chance of being awake. Similarly, model 362 may output a confidence score p2 indicating a confidence (probability) of being in “N1-N2” stage but not “awake” state, model 363 may output a confidence score p3 indicating a confidence (probability) of being in “N3” stage but not “N1-N2” stage, model 364 may output a confidence score p4 indicating a confidence (probability) of being in REM phase but not “N3” stage of non-REM sleep, and mode 365 may output a confidence score p5 indicating a confidence (probability) of being in non-REM phase but not in REM phase of sleep. The numerical value p can be determined depending on the loss function defined by the ML-based classification model. One example of such loss function is a cross-entropy loss function, which produces a numerical output between zero and one. In an example, the ML-based classification models 360 employ fuzzy-logic classification to output the numerical value p.

The regression model 370 can map the model-specific awake or sleep classes produced by the ML-based classification models 360 to determine a composite awake or sleep classification. In an example, the regression model 370 is a logistic regression model. In an example, the regression model 370 can be trained to determine respective weights for confidence scores associated with each of the awake or sleep classes determined from the ML-based classification models 360, compute a weighted combination of the confidence scores each weighted by the respective weights, and compare the weighed combination to one or more threshold value to determine the composite awake or sleep classification. The combination can be a linear combination in one example, or a non-linear combination in another example. In the example illustrated in FIG. 3, to make a binary classification (e.g., sleep vs. awake), a logistic regression model can be constructed using the confidence scores p1-p5 produced respectively by ML-based classification models 361-365:

$$P = 1 / exp ( {w}_{0} + {\sum }_{i = 1}^{5} {w}_{i} {p}_{i} )$$

Where w₀ is a bias constant and w₁-w₅ are weights for respective confidence scores p1-p5. The bias w₀ and the weights w₁-w₅ can be determined through model training. The model output P can be compared to a threshold (P_{TH}) to determine a composite classification of either an “awake” state or a “sleep” state for the patient.

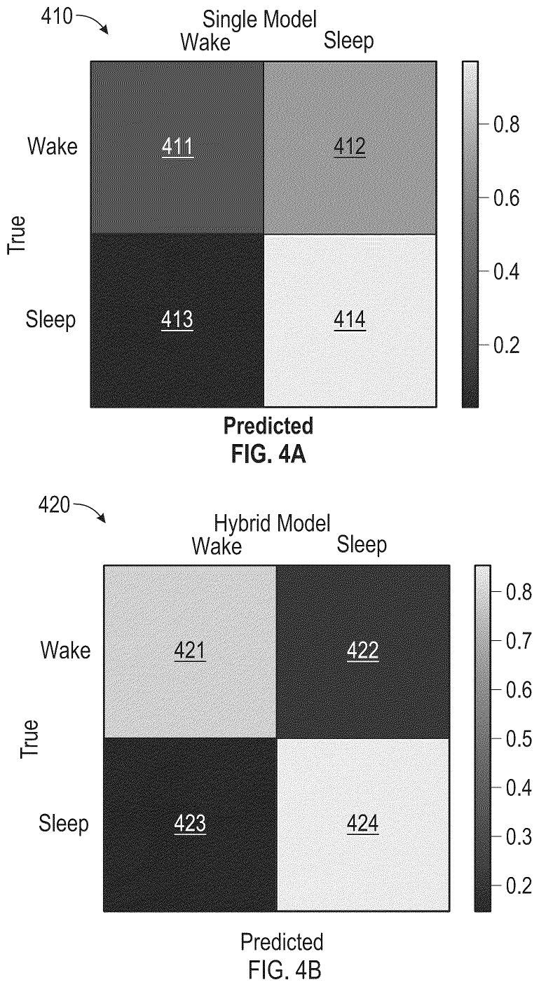

By combining the classifications from the ML-based classification models 360, the regression model 370 can improve the sensitivity and specificity of sleep detection. FIGS. 4A-4B illustrate example confusion matrices showing sleep classification performances of a single sleep detection model and a hybrid sleep detection model such as the sleep detection and classification model 352. The confusion matrix 410 in FIG. 4A is constructed using classification results produced by a single model such as model 361. The confusion matrix 420 in FIG. 4B is constructed using classification results produced by the hybrid sleep detection and classification model 352. In the illustrated example, the classification is between awake and sleep states. The sleep state is defined as a “positive” state, and the awake state is defined as a “negative” state. As illustrated, the two models show similar true positive (TP, actual “sleep” state correctly detected by the model as a “sleep” state) rates 414 and 424 of approximately 0.9, and also similar false negative (FN, actual “sleep” state incorrectly detected by the model as an “awake” state) 413 and 423 of approximately 0.1. However, the two models show significantly difference in true negative (TN, actual “awake” state correctly detected by the model as an “awake” state) rate and in false positive (FP, actual “awake” state incorrectly detected by the model as a “sleep” state). For example, while the single model shows a TN rate 411 of approximately 0.3 and a FP rate 412 of approximately 0.7, the hybrid model produced a significantly higher TN rate 421 of approximately 0.8 and a significantly lower FP rate 422 of approximately 0.2. With the lower FP rate, the hybrid sleep detection and classification model improves the specificity of sleep detection.

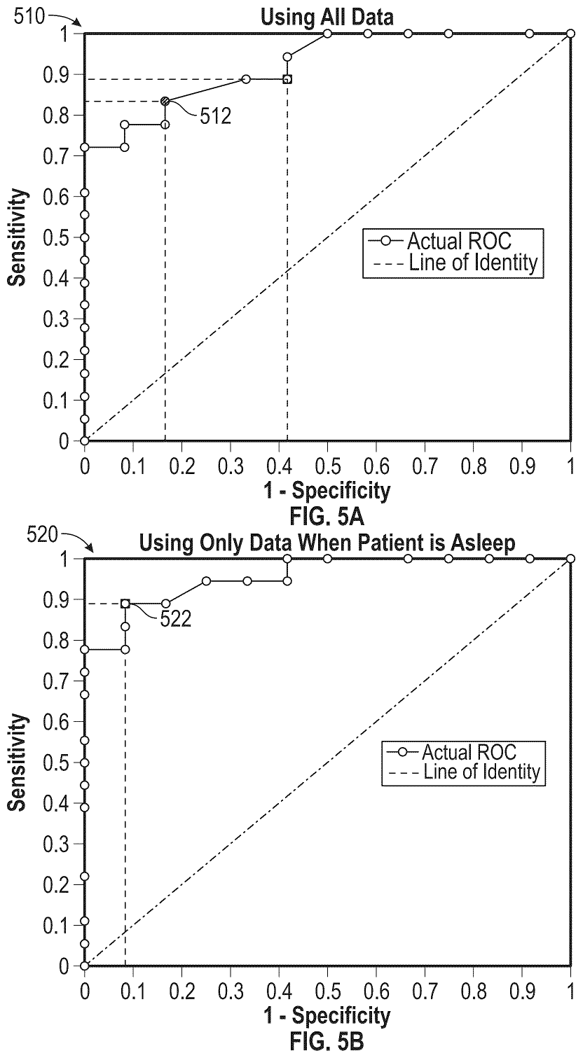

As described above, the determination of sleep state and sleep stage may be used for sleep apnea diagnosis or other context-aware diagnosis. FIGS. 5A-5B are graphs of receiver operating characteristic (ROC) curves showing sleep apnea detection performances with and without confirmed sleep state of the patient, which may be detected using the trained hybrid sleep detection and classification model 352. ROC curve 510 in FIG. 5A shows a performance of apnea detection when all physiological data, regardless of sleep states or sleep stage, are used to detect sleep apnea. ROC curve 520 in FIG. 5B shows a performance of apnea detector that uses only the physiological data collected during confirmed sleep state of the patient, such as determined using the hybrid sleep detection and classification model 352. Each ROC curve shows sleep apnea detection sensitivity values (on the y-axis) and false positive rate (on the x-axis, which is equivalent to 1-specficity) at different detection threshold values. By way of example and not limitations, two metrics derived from the ROC curve may be used to evaluate sleep detection performances. The first metric is area under the ROC curve (AUC), which represents an aggregate measure of performance across all possible classification thresholds. The second metric is optimal operating point representing a desired sensitivity and specificity combination. As shown in FIGS. 5A-5B, the ROC curve 520 has a higher AUC (AUC=0.95) than the ROC curve 510 (AUC=0.91). An optimal operating point 512 on the ROC curve 510 corresponds to a sensitivity of 0.833 and a specificity 0.833. In contrast, an optimal operating point 522 identified from ROC curve 520 corresponds to a sensitivity of 0.889 and a specificity of 0.917. Both the AUC and optimal operation point metrics suggest that incorporating a confirmed sleep state into apnea detection, such as by using only the physiological data collected during confirmed sleep (e.g., as determined using the hybrid sleep detection and classification model as described herein) can substantially improve apnea detection performance.

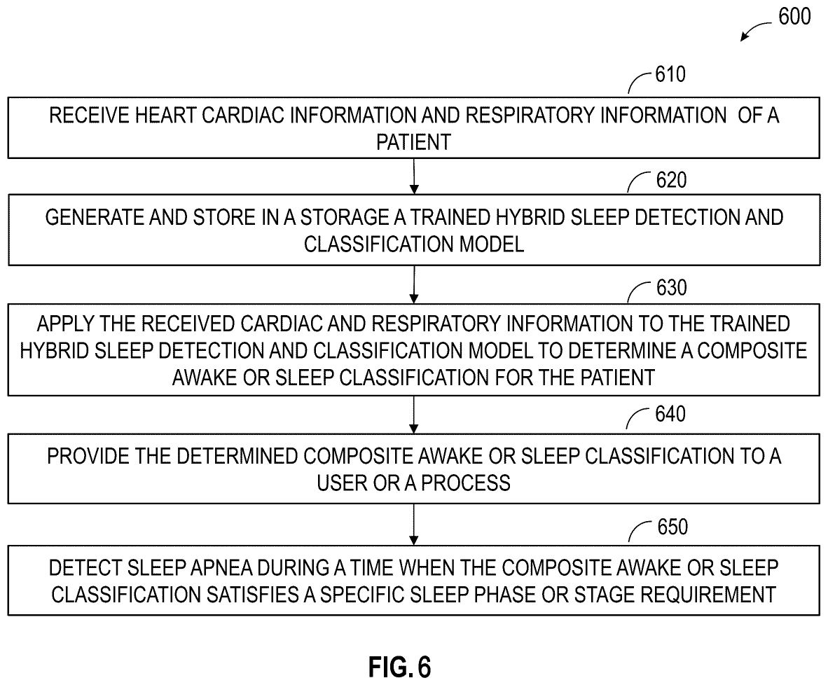

FIG. 6 is a flowchart illustrating an example method 600 of monitoring and staging sleep in a patient. The method 600 may be implemented and executed in an ambulatory medical device such as an implantable or wearable medical device, or in a remote patient management system. In an example, the method 600 may be implemented in and executed by the IMD 102 or the WMD 103, the external system 105, or the sleep detection and staging system 200.

The method 600 begins at step 610, where cardiac information and respiratory information of a patient may be received such as using the data receiver circuit 210. The cardiac information may include ECG or EGM, or signals indicative of cardiac mechanical activity, such as pressure, impedance, heart sounds, or respiration signals. The respiration information may include a tidal volume, a respiration rate, a minute ventilation, a respiratory sound, or a rapid-shallow breathing index (RSBI) computed as a ratio of a respiratory rate measurement to a tidal volume measurement, among others. The sensed cardiac and respiration information may be pre-processed, including amplification, digitization, filtering, or other signal conditioning operations. In some examples, the cardiac and respiration information may be sensed and stored in a storage device, such as an electronic medical record system, and retrieved for use upon a user or a system request.

At 620, a trained hybrid sleep detection and classification model may be generated and stored in a storage device. As described above with respect to FIG. 3, the trained hybrid sleep detection and classification model may comprise a plurality of trained machine-learning (ML) models and a trained regression model. The trained ML models are each trained to map input cardiac and respiratory data into one of multiple distinct model-specific awake or sleep classes. Examples of model-specific awake or sleep classes include an awake state, a sleep state, a rapid eye movement (REM) phase of sleep, a non-REM phase of sleep, an N1 stage of non-REM sleep, an N2 stage of non-REM sleep, a combined N1-N2 stage of non-REM sleep, or a N3 stage of non-REM sleep, among others. The ML-based classification models may each have a specific model architecture (e.g., a decision tree, a random forest, a neural network, a deep neural network, etc.), and be trained using a training dataset that includes cardiac and respiratory information collected from a patient population during known awake or sleep states or sleep phases or stages. The ML-based classification models may be trained using supervised learning or unsupervised learning.

The regression model may be trained to map the model-specific awake or sleep classes produced by the plurality of ML models to a composite awake or sleep classification. In an example, the trained regression model is a logistic regression model. In some examples, the ML-based classification models may each be trained to output a confidence score associated with the model-specific awake or sleep classification determined for the cardiac and respiration information input. The confidence score can be probability (taking value between 0 and 1) indicating how likely the patient is in the classified state or sleep phase or stage. The regression model can be trained to determine respective weights for confidence scores associated with each of the awake or sleep classes determined from the ML-based classification models, compute a weighted combination of the confidence scores each weighted by the respective weights, and compare the weighed combination to one or more threshold value to determine the composite awake or sleep classification. The weighted combination can be a linear combination, or a non-linear combination.

At 630, the cardiac and respiratory information received from step 610 can be applied to the trained hybrid sleep detection and classification model to determine a corresponding composite awake or sleep classification for the patient. This may include applying the received cardiac and respiratory information to each of the plurality of trained ML models to determine a model-specific awake or sleep classification and a confidence score associated with the determined model-specific awake or sleep classification, and applying the confidence scores associated with the determined awake or sleep classes to the trained regression model to determine the corresponding composite awake or sleep classification for the patient. In an example, the composite awake or sleep classification can be determined by comparing the weighted combination of the confidence scores associated with each of the awake or sleep classes determined from the ML-based classification models to one or more threshold values.

At 640, the determined composite awake or sleep classification may be provided to a user or a process executable by the medical-device system. In some examples, a physiological event can be detected, or a context-aware diagnosis can be made, using the composite awake or sleep classification. At 650, sleep apnea can be detected during a time when the composite awake or sleep classification satisfies a specific sleep phase or stage requirement. For example, only the physiological information collected during a time when the patient is determined to be in a sleep state or in one or more specific sleep phases and/or stages is used to detect sleep apnea. Improved sleep apnea diagnosis performance can be achieved when using only physiological information collected during sleep as detected by a hybrid sleep detection and classification model, as described above with respect to FIGS. 4A-4B.

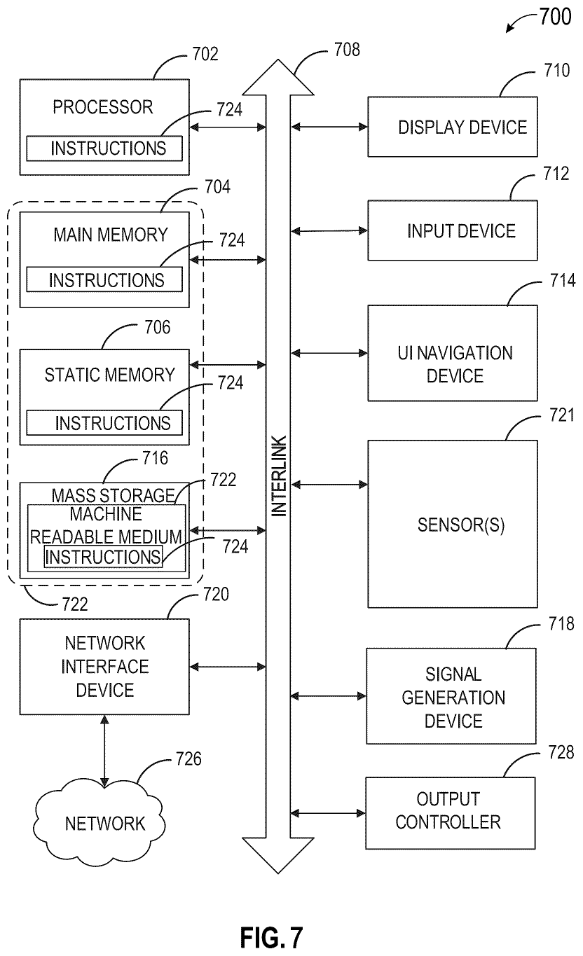

FIG. 7 illustrates generally a block diagram of an example machine 700 upon which any one or more of the techniques (e.g., methodologies) discussed herein may perform. Portions of this description may apply to the computing framework of various portions of the patient management system 100 or of the sleep detection and staging system 200.

In alternative embodiments, the machine 700 may operate as a standalone device or may be connected (e.g., networked) to other machines. In a networked deployment, the machine 700 may operate in the capacity of a server machine, a client machine, or both in server-client network environments. In an example, the machine 700 may act as a peer machine in peer-to-peer (P2P) (or other distributed) network environment. The machine 700 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate by, logic or a number of components, or mechanisms. Circuit sets are a collection of circuits implemented in tangible entities that include hardware (e.g., simple circuits, gates, logic, etc.). Circuit set membership may be flexible over time and underlying hardware variability. Circuit sets include members that may, alone or in combination, perform specified operations when operating. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

Machine (e.g., computer system) 700 may include a hardware processor 702 (e.g., a central processing unit (CPU), a graphics processing unit (GPU), a hardware processor core, or any combination thereof), a main memory 704 and a static memory 706, some or all of which may communicate with each other via an interlink (e.g., bus) 708. The machine 700 may further include a display unit 710 (e.g., a raster display, vector display, holographic display, etc.), an alphanumeric input device 712 (e.g., a keyboard), and a user interface (UI) navigation device 714 (e.g., a mouse). In an example, the display unit 710, input device 712 and UI navigation device 714 may be a touch screen display. The machine 700 may additionally include a storage device (e.g., drive unit) 716, a signal generation device 718 (e.g., a speaker), a network interface device 720, and one or more sensors 721, such as a global positioning system (GPS) sensor, compass, accelerometer, or other sensors. The machine 700 may include an output controller 728, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 716 may include a machine-readable medium 722 on which is stored one or more sets of data structures or instructions 724 (e.g., software) embodying or utilized by any one or more of the techniques or functions described herein. The instructions 724 may also reside, completely or at least partially, within the main memory 704, within static memory 706, or within the hardware processor 702 during execution thereof by the machine 700. In an example, one or any combination of the hardware processor 702, the main memory 704, the static memory 706, or the storage device 716 may constitute machine-readable media.

While the machine-readable medium 722 is illustrated as a single medium, the term “machine-readable medium” may include a single medium or multiple media (e.g., a centralized or distributed database, and/or associated caches and servers) configured to store the one or more instructions 724.

The term “machine-readable medium” may include any medium that is capable of storing, encoding, or carrying instructions for execution by the machine 700 and that cause the machine 700 to perform any one or more of the techniques of the present disclosure, or that is capable of storing, encoding or carrying data structures used by or associated with such instructions. Non-limiting machine-readable medium examples may include solid-state memories, and optical and magnetic media. In an example, a massed machine-readable medium comprises a machine-readable medium with a plurality of particles having invariant (e.g., rest) mass. Accordingly, massed machine-readable media are not transitory propagating signals. Specific examples of massed machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 724 may further be transmitted or received over a communications network 726 using a transmission medium via the network interface device 720 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as WiFi®, IEEE 802. 16 family of standards known as WiMax®), IEEE 802. 15. 4 family of standards, peer-to-peer (P2P) networks, among others. In an example, the network interface device 720 may include one or more physical jacks (e.g., Ethernet, coaxial, or phone jacks) or one or more antennas to connect to the communications network 726. In an example, the network interface device 720 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 700, and includes digital or analog communications signals or other intangible medium to facilitate communication of such software.

Various embodiments are illustrated in the figures above. One or more features from one or more of these embodiments may be combined to form other embodiments.

The method examples described herein can be machine or computer-implemented at least in part. Some examples may include a computer-readable medium or machine-readable medium encoded with instructions operable to configure an electronic device or system to perform methods as described in the above examples. An implementation of such methods may include code, such as microcode, assembly language code, a higher-level language code, or the like. Such code may include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A medical-device system for monitoring and staging sleep in a patient, the system comprising:
a receiver circuit configured to receive cardiac information and respiratory information of the patient;
a storage device configured to store a trained hybrid sleep detection and classification model comprising:
a plurality of trained machine-learning (ML) models each trained to map cardiac and respiratory data into one of multiple distinct model-specific awake or sleep classes; and
a trained regression model trained to map the model-specific awake or sleep classes produced by the plurality of ML models to a composite awake or sleep classification; and
a controller circuit, comprising a sleep detector configured to apply the received cardiac and respiratory information to the trained hybrid sleep detection and classification model to determine a corresponding composite awake or sleep classification for the patient;
wherein the controller is configured to provide the determined composite awake or sleep classification to a user or a process executable by the medical-device system.

2. The medical-device system of claim 1, wherein to apply the received cardiac and respiratory information to the trained hybrid sleep detection and classification model to determine the corresponding composite awake or sleep classification, the controller circuit is configured to:
apply the received cardiac and respiratory information to each of the plurality of trained ML models to determine a model-specific awake or sleep classification and a confidence score associated with the determined model-specific awake or sleep classification; and
apply the confidence scores associated with the determined awake or sleep classes to the trained regression model to determine the corresponding composite awake or sleep classification for the patient.

3. The medical-device system of claim 1, wherein the receiver circuit is electrically coupled to a cardiac sensor configured to sense the cardiac information of the patient including a surface or subcutaneous electrocardiogram or heart sound information.

4. The medical-device system of claim 1, wherein the receiver circuit is electrically coupled to a respiratory sensor configured to sense the respiratory information of the patient including a respiratory rate or a tidal volume.

5. The medical-device system of claim 1, wherein the plurality of trained ML models include binary classification models each trained to map the cardiac and respiratory data into one of two model-specific awake or sleep classes.

6. The medical-device system of claim 1, wherein the plurality of trained ML models include one or more trained decision tree models, one or more trained random forest models, or one or more neural network or deep neural network models.

7. The medical-device system of claim 1, wherein the model-specific awake or sleep classes include one or more of an awake state or a sleep state.

8. The medical-device system of claim 1, wherein the model-specific awake or sleep classes include one or more sleep phases or stages selected from the group consisting of:
a rapid eye movement (REM) phase of sleep;
a non-REM phase of sleep;
an N1 stage of non-REM sleep;
an N2 stage of non-REM sleep;
a combined N1-N2 stage of non-REM sleep; and
an N3 stage of non-REM sleep.

9. The medical-device system of claim 1, wherein the trained regression model is trained to determine respective weights for confidence scores associated with the determined awake or sleep classes, and to determine the composite awake or sleep classification using a weighted combination of the confidence scores each weighted by the respective weights.

10. The medical-device system of claim 1, wherein the trained regression model is a logistic regression model.

11. The medical-device system of claim 1, wherein the controller circuit includes a training module configured to generate the trained hybrid sleep detection and classification model, including:
to generate the plurality of trained ML models using a first training dataset comprising input cardiac and respiratory data collected from a group of patients during known awake or sleep states or sleep stages; and
to generate the trained regression model using a second training dataset comprising awake or sleep classifications and confidence scores associated with the awake or sleep classifications and the known awake or sleep states or sleep stages.

12. The medical-device system of claim 1, comprising an apnea detector circuit configured to detect sleep apnea during a time when the composite awake or sleep classification satisfies a specific sleep phase or stage requirement.

13. A method of monitoring and staging sleep in a patient using a medical-device system, the method comprising:
receiving cardiac information and respiratory information of the patient;
generating and storing in a storage device a trained hybrid sleep detection and classification model comprising (i) a plurality of trained machine-learning (ML) models each trained to map cardiac and respiratory data into one of multiple distinct model-specific awake or sleep classes, and (ii) a trained regression model trained to map the model-specific awake or sleep classes produced by the plurality of ML models to a composite awake or sleep classification;
applying, via a sleep detector, the received cardiac and respiratory information to the trained hybrid sleep detection and classification model to determine a corresponding composite awake or sleep classification for the patient; and
providing the determined composite awake or sleep classification to a user or a process executable by the medical-device system.

14. The method of claim 13, wherein applying the received cardiac and respiratory information to the trained hybrid sleep detection and classification model to determine the corresponding composite awake or sleep classification includes:
applying the received cardiac and respiratory information to each of the plurality of trained ML models to determine a model-specific awake or sleep classification and a confidence score associated with the determined model-specific awake or sleep classification; and
applying the confidence scores associated with the determined awake or sleep classes to the trained regression model to determine the corresponding composite awake or sleep classification for the patient.

15. The method of claim 13, wherein the trained regression model is trained to determine respective weights for confidence scores associated with the determined awake or sleep classes, and to determine the composite awake or sleep classification using a weighted combination of the confidence scores each weighted by the respective weights.

16. The method of claim 13, wherein:
the received cardiac information includes a surface or subcutaneous electrocardiogram or heart sound information; and
the received respiratory information includes a respiratory rate or a tidal volume.

17. The method of claim 13, wherein the plurality of trained ML models include binary classification models each trained to map the cardiac and respiratory data into one of two model-specific awake or sleep classes.

18. The method of claim 13, wherein the plurality of trained ML models include one or more trained decision tree models, one or more trained random forest models, or one or more neural network or deep neural network models.

19. The method of claim 13, wherein the model-specific awake or sleep classes include one or more of:
an awake state;
a sleep state;
a rapid eye movement (REM) phase of sleep;
a non-REM phase of sleep;
an N1 stage of non-REM sleep;
an N2 stage of non-REM sleep;
a combined N1-N2 stage of non-REM sleep; and
an N3 stage of non-REM sleep.

20. The method of claim 13, comprising an apnea detector circuit configured to detect sleep apnea during a time when the composite awake or sleep classification satisfies a specific sleep phase or stage requirement.
